(19) United States
(12) Patent Application Publication
(10) Pub. No.: US 2025/0235684 A1
(43) Pub. Date: Jul. 24, 2025

(54) CONNECTOR COMPRISING AN ANTI-ROTATION AND ANTI-DISPLACEMENT MEANS

(71) Applicant: B. Braun Melsungen AG, Melsungen (DE)

(72) Inventors: Jens Joern, Kassel (DE); Florin Kopp, Schortens (DE); Johannes Bolz, Kassel (DE); Christian Hohmann, Edermuende (DE)

(21) Appl. No.: 18/709,921

(22) PCT Filed: Nov. 11, 2022
(86) PCT No.: PCT/EP2022/081582

§ 371 Date: May 14, 2024

(30) Foreign Application Priority Data
Nov. 15, 2021 (DE) 10 2021 129 674.9

(51) Int. Cl. A61M 39/10; A61M 39/12

(52) U.S. Cl. CPC A61M 39/1011; A61M 39/12; A61M 2039/1066; A61M 2039/1077; A61M 2205/0216; A61M 2205/586

(57) ABSTRACT

A connector for establishing a fluidic connection between a first fluid transmission component and a second fluid transmission component includes a connecting piece having an internal thread and to which a corresponding counter-piece is/can be screwed. A union sleeve can be slid over the connector, in which state the union sleeve engages with the connecting piece in a non-detachable manner. A latching mechanism of the union sleeve, when slid over, engages with an end face of the connection piece that faces the counter-piece.

### CROSS-REFERENCE TO RELATED APPLICATIONS

This application is the United States national stage entry of International Application No. PCT/EP2022/081582, filed on Nov. 11, 2022, and claims priority to German Application No. 2021 129 674.9, filed on Nov. 15, 2021. The contents of International Application No. PCT/EP2022/081582 and German Application No. 10 2021 129 674.9 are incorporated by reference herein in their entireties.

### FIELD

The invention relates to a connector for establishing a fluid connection between a first and a second fluid transmission component, having an adapter piece which has an internal thread and to which a corresponding counter-piece is screwed/can be screwed, and a union sleeve which is slidable over the connector and which, in a slid-on state, latches non-detachably with the adapter piece.

### BACKGROUND

In infusion therapy, the use of Luer and Luer-Lock connections is well known for connecting fluid transmission components, such as infusion lines, extensions and branches, to each other and to the patient. These connections are usually configured to be detachable. However, in some cases, in particular when handling toxic substances, such as in cancer therapy, it is desirable not to be able to reopen connections once they have been closed, for example in order to avoid contamination. Such non-detachable connections therefore have to be compatible with Luer connections in order to make the connection, but must not be able to be detached (non-destructively) once the connection has been made.

Non-detachable connectors for infusion sets are known from the prior art. EP 3 530 312 A1, for example, discloses a non-detachable connector for supply lines for intravenous infusions. The connector has an adapter piece and a union sleeve, wherein both have matching latching means that latch the adapter piece and the union sleeve together and secure them axially against each other. This prevents relative movement between the union sleeve and the adapter piece in the axial direction.

EP 3 081 253 A1 discloses an adapter piece with a connector body and a lock nut/union sleeve. The connector body has a male Luer connection for connecting the adapter piece to a corresponding counter-piece. The connector body is also configured to be screwed to the lock nut in order to clamp a tube portion attached to an end portion of the connector body. Furthermore, interlocking anti-rotation means are provided on the connector body and the lock nut, which prevent relative movement between the lock nut and the connector body in the circumferential direction when the tube is clamped. This ensures a non-destructively detachable connection.

Furthermore, EP 1 917 996 B1 also shows a non-reconnectable positive Luer-Lock adapter piece for connection to all standardized negative Luer-Lock connections. The adapter piece has a toothing which is brought into engagement with a mating toothing of a rotatable union sleeve when the union sleeve is placed over the adapter piece. The toothing and the mating toothing are configured in such a way that they engage in a locking manner in a first rotational direction to lock the Luer lock connection of the union sleeve and slide freely against each other in a second rotational direction opposite to the first. For this purpose, latching means are formed on the adapter piece and/or on the union sleeve for non-detachable latching of the union sleeve with the adapter piece.

In addition, DE 20 2004 015 957 Ul discloses a sleeve which can be used to fix and seal a coupling, e.g. a Luer-Lock coupling, between two adapter pieces. For this purpose, the sleeve has molded parts which cover and stabilize the adapter pieces after they have been coupled.

The solutions known from the prior art have the following disadvantage. The union sleeves known from the prior art have specific latching means which correspond to the corresponding latching means of the connectors or adapter pieces. This means that the union sleeves have a complex design and are not suitable for different connectors.

### SUMMARY

The objects and objectives of the invention are to eliminate or at least reduce the disadvantages of the prior art and, in particular, to provide a union sleeve for a connector which is easy and inexpensive to manufacture and can be used universally and which prevents or impedes detaching of a connection between two fluid transmission components.

The objects and objectives are solved with regard to a generic connector, based on the realization of using the geometries of the connector components to secure the connector.

According to the invention, the connector is configured/adapted so that a latching means of the union sleeve latches in the slid-on state with an axially end-side front face of the adapter piece facing the counter-piece. In this way, access to the connector in the connected state can be prevented or made more difficult by the union sleeve without the need for special/specific latching means, for example in the form of recesses, which impair the structural integrity of the connector. Since no specific latching means are required for the adapter piece, but the axially end-side front face of the adapter piece itself serves as the latching geometry for the latching means, the union sleeve can be used for a large number of connectors.

Advantageous embodiments are explained below.

In a preferred variant, the adapter piece may have a sleeve part with the internal thread and a conical part housed in the sleeve part and connected to a tube portion of the first fluid transmission component. Alternatively, the adapter piece may also be configured in one piece, i.e. the sleeve part and the conical part are configured as integral/one-piece components.

According to an advantageous further development, the union sleeve may have partially circumferential projections in the axial direction/extension direction of the union sleeve on an end portion facing the counter-piece, wherein in the slid-on state the projections are in engagement with the connector, in particular with a connector handle. It is also conceivable that the connector handle has a circular cylindrical main body, at least one first adapter portion branching off from the main body and being in fluid connection with the counter-piece, and two handle portions projecting from an outer circumferential surface of the main body, between which the partially circumferential projections of the union sleeve are held in the slid-on state of the union sleeve.

Furthermore, it may be expedient if the connector handle has a further second adapter portion branching off from the main body and being couplable to the second fluid transmission component. According to the invention, it may be advantageous if a central axis of the first adapter portion is parallel to a central axis of the second adapter portion.

In a preferred embodiment, the latching means of the union sleeve may be pre-tensioned by the connector, in particular the adapter piece, when the union sleeve is slid onto the connector.

According to a particularly preferred embodiment, the union sleeve may have as a latching means at least one elastic latching hook/tab projecting axially and radially inward from an inner circumferential surface of the union sleeve. In particular, it is advantageous if the union sleeve has three latching hooks/tabs evenly distributed over the circumference.

Furthermore, it is conceivable that the union sleeve may have at least one handle element on its outer circumferential surface, which can be gripped when the union sleeve is slid on by a user.

According to an advantageous embodiment, the at least one latching hook may have an axial latching portion, which is configured to latch with the front face facing the counter-piece in the slid-on state, and a radial latching portion, which is configured to engage behind or rest against an outer circumferential surface of the adapter piece in the slid-on state. In other words, the at least one latching hook may have the axial latching portion and the radial latching portion in order to fix the at least one latching hook in the axial direction or in the slide-on direction and in the radial direction relative to the adapter piece. This prevents that the at least one latching hook slips radially inward on the front face of the adapter piece facing the counter-piece, thus engaging in the adapter piece and shearing off in the process when a tensile load is applied against the slide-on direction.

In yet other words, the latching hooks may be mounted at one point inside the union sleeve according to the advantageous further development. There are no apertures to the outside in the lateral surface of the union sleeve. The latching hooks are arranged in such a way that they are self-locking under tensile load and do not slide off over the adapter piece (nut) in order to prevent the union sleeve from being dismantled. The latching hooks are provided with an additional lug (radial latching portion) that rests on the adapter piece. This prevents the latching hook from slipping under the counter-piece under tensile load. Higher tensile forces can be absorbed and the shearing of the latching hook can be counteracted.

In a further advantageous further development, the at least one latching hook may be mounted at two points in the union sleeve, wherein the union sleeve for the at least one latching hook has at least two apertures formed on the side of the latching hook, which ensure elastic deflection of the at least one latching hook when the union sleeve is pushed on or mounted.

In other words, in this advantageous further development, the latching hooks may be mounted at two points inside the union sleeve. The spring effect for deflecting the latching hook during assembly of the union sleeve is achieved via clearances/apertures in the lateral surface of the union sleeve. The latching hooks are also arranged in such a way that they are self-locking under tensile load and do not slide off over the adapter piece in order to prevent the union sleeve from being dismantled. This variant also prevents the latching hook from slipping under the counter-piece under tensile load. Higher tensile forces can be absorbed and the shearing of the latching hook can be counteracted.

In other words, the invention relates to a cover element for a plurality of connector types, which is slidable over the connection of the connector and, in the latched state, prevents detaching of the connection. This is achieved by elastic latching means on the circumference of the inner surface of the cover element, which engage on a front face of the connector in the latched state and thus secure the union sleeve against axial displacement on the connector. In order to prevent rotation, the cover element has partially circumferential projections in the axial direction at one end, which interact with a handle of the connector attachment when the cover element is latched and largely prevent the cover element from rotating around the connector.

### BRIEF DESCRIPTION OF THE DRAWINGS

The invention is explained in more detail below with reference to preferred configuration examples with the aid of Figures.

FIG. 1 shows a perspective view of a connector according to a preferred configuration example;

FIG. 2 shows a sectional view of the connector according to the preferred configuration example;

FIG. 3 shows a perspective view of a union sleeve according to the preferred configuration example;

FIG. 4 shows a top view of the union sleeve according to the preferred configuration example;

FIG. 5 shows a perspective view of a union sleeve according to a modification of the preferred configuration example;

FIG. 6 shows a top view of the union sleeve according to the modification of the preferred configuration example;

FIG. 7 shows a sectional view of a connector according to another preferred configuration example;

FIG. 8 shows a sectional view of a union sleeve according to the further preferred configuration example; and

FIG. 9 shows a sectional view of a connector according to a modification of the further preferred configuration example.

The Figures are schematic in nature and are provided solely for the purpose of understanding the invention. Identical elements are marked with the same reference signs. The features of the various configuration examples or modifications can be interchanged.

### DETAILED DESCRIPTION

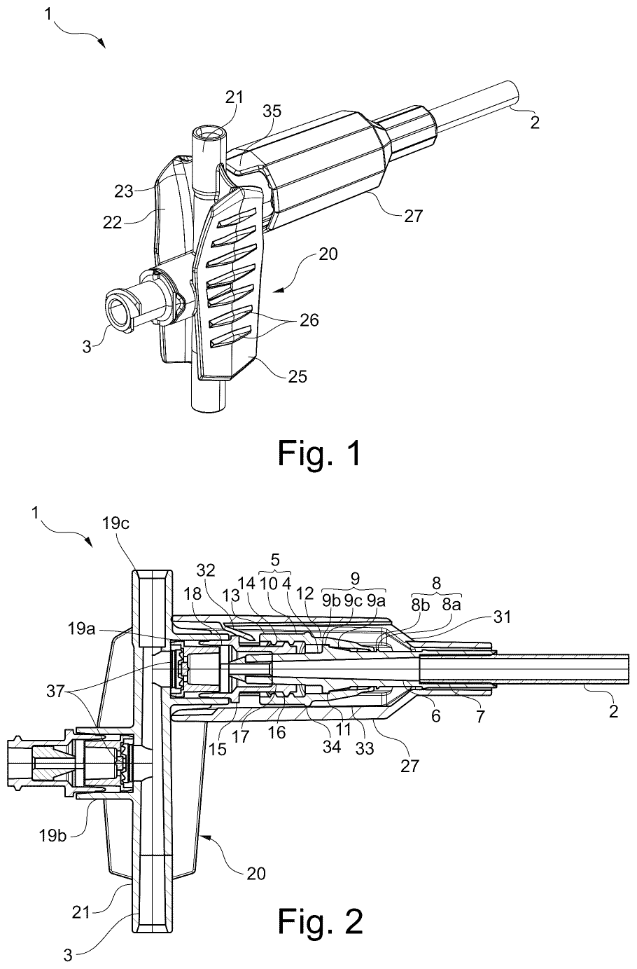

FIG. 1 shows a connector 1 for establishing a fluid connection between two fluid transmission components. The fluid transmission components may be, for example, an ampoule, a syringe or a tube or a tube portion. In the configuration example shown in FIG. 1, the connector 1 is connected on the one hand to a tube portion 2 as the first fluid transmission component, which in turn can be connected to a further fluid transmission component. Furthermore, the connector 1 has an adapter portion 3, which can be connected to the further fluid transmission component, in particular a further tube portion.

As shown in FIG. 2, the tube portion 2 is housed in a conical part 4 of an adapter piece 5 of the connector 1. The conical part 4 is substantially configured as a conical cylinder. This means that a fluid opening 6 extends through the conical part 4 in the axial direction, wherein an outer diameter and an inner diameter of the conical part 4 decrease from a side facing the tube portion 2 to a side facing away from the tube portion 2. At the end portion facing the tube portion 2, the conical part 4 has an insertion portion 7 with an enlarged inner diameter as compared to the fluid opening 6. In other words, the insertion portion 7 is offset from the fluid opening 6 by a step in the radial direction toward the outside. When the tube portion 2 is slid into the insertion portion 7, this step serves as an axial stop. In order to fix the tube portion 2 in the insertion portion 7, the inner diameter of the insertion portion 7 in the preferred configuration example is smaller than an outer diameter of the tube portion 2, so that the tube portion 2 is held in the insertion portion 7. Alternatively or additionally, the insertion portion 7 may also have a circumferential nose or a projecting portion, which clamps the tube portion 2 and thus holds it in the insertion portion 7. Furthermore, the tube portion 2 may additionally or alternatively be glued into the insertion portion 7, i.e. the tube portion 2 and the insertion portion 7 may be connected via an adhesive bond, in particular with a solvent-based adhesive.

A first latching portion 8 and a second latching portion 9 are formed on an outer circumferential surface of a central section of the conical part 4. The first latching portion 8 is in the form of a circumferential ramp with a ramp surface 8a facing the tube portion 2 and a latching surface 8b aligned substantially perpendicular to the axial direction of the conical part 4. The second latching portion 9 also has the shape of a circumferential ramp, although its dimensions are larger than those of the first latching portion 8 in both the axial and radial directions. In other words, a ramp surface 9a and a latching surface 9b of the second latching portion 9 are larger than the ramp surface 8a and the latching surface 8b of the first latching portion 8. In other words, the cross-sectional area defined by the second latching portion 9 is larger than the cross-sectional area defined by the first latching portion 8. Furthermore, a step 9c is formed on a ramp surface 9a of the second latching portion 9

As shown in FIG. 2, a step 11 formed on an inner circumferential surface of a sleeve part 10 of the adapter piece 5 is supported on this step 9c. The sleeve part 10 is substantially in the form of a sleeve with a connection portion 12 facing the tube portion 2 and a threaded portion 13 facing away from the tube portion 2. The connection portion 12 is conically tapered, i.e. with an outer diameter that decreases in the axial direction toward the tube portion 2. The threaded portion 13 has an internal thread 14, in particular a Luer thread, to which a counter-piece 15, described in more detail below, is screwed.

As can be seen in FIG. 2, an end portion of the connection portion 12 is configured with a smaller inner diameter than a maximum outer diameter of the first latching portion 8. If the sleeve part 10 is now moved in the axial direction relative to the conical part 4, or is pushed over the conical part 4, the end portion of the connection portion 12 engages with the first latching portion 8 and the step 11 of the connection portion 12 rests against the step 9c of the conical part 4, i.e. the conical part 4, in particular the connection portion 12, is held between the first latching portion 8 and the second latching portion 9. The conical part 4 and the sleeve part 10 thus form the adapter piece 5.

As mentioned above, the threaded portion 13 of the adapter piece 5 is screwed to the counter-piece 15. For this purpose, the counter-piece 15 has an outer thread 16, which is screwed to the internal thread 14. An inner circumferential surface 17 of the counter-piece 15 is conical, so that when the counter-piece 15 is screwed to the adapter piece 5, the conical part 4 is in fluid-tight contact with the inner circumferential surface 17 in order to establish a fluid connection between the adapter piece 5 and the counter-piece 15.

At an end portion facing away from the adapter piece 5, the counter-piece 15 furthermore has a receiving opening 18, in which an adapter portion 19a of a connector handle 20 is housed. As can be seen in FIG. 2, the connector handle 20 has a cylindrical main body 21 and the two adapter portions 19a, 19b arranged branching off from it. This means that fluid from the tube portion 2 as the first fluid transmission component flows through the fluid opening 6 of the conical part 4 of the adapter piece 5 into the counter-piece 15 and from there via the adapter portion 19a into the cylindrical main body 21, where it can finally flow via the adapter portion 3 into the second fluid transmission component connected to the adapter portion 3. Of course, a fluid flow from the second fluid transmission component in reverse order through the connector 1 to the first fluid transmission component, i.e. the tube portion 2, is also conceivable. Furthermore, the main body 21 has a third adapter portion 19c on an end portion opposite the adapter portion 3 in the axial direction, which can be connected accordingly to a further fluid transmission component, in particular a further tube portion. Substantially, this results in two flow paths for the connector 1 according to the preferred configuration examples. A first main flow path extends, as mentioned above, from the tube portion 2 via the fluid opening 6 of the adapter piece 5, the counter-piece 15, the adapter portion 19a, the cylindrical main body 21 to the adapter portion 3 and the fluid transmission component connected to it. A second flow path is formed between the adapter portion 19b and the adapter portion 19c or between the fluid transmission components connected to these adapter portions 19b and 19c. As can be seen in FIG. 2, the adapter portion 19b is substantially identical to the adapter portion 19a. This means that the adapter portion 19b has a counter-piece which can be screwed to a further adapter piece.

A check valve 37 is arranged in each of the adapter portions 19a, 19b to prevent backflow or to ensure separation of the two flow paths.

In the preferred configuration example of the connector 1 shown in FIG. 2, the adapter portions 19a, 19b are arranged on the main body 21 such that they branch off substantially perpendicularly therefrom. That is, a central axis of the adapter portion 19a and a central axis of the adapter portion 19b are substantially perpendicular to a central axis of the main body 21. In other words, the central axis of the adapter portion 19a is parallel to the central axis of the adapter portion 19b, so that the adapter portions 19a, 19b branch off perpendicularly from the main body 21 at diametrically opposite locations of the main body 21 that are offset in an axial direction of the main body 21.

As shown in FIG. 1, the connector handle 20 has two handle portions 22, which extend in the axial direction of the main body 21 and project from the main body 21 at two diametrically opposite locations. In particular, the handle portions 22 each have a handle bar 23 and a handle plate 24. As shown in FIG. 1, the handle bars 23 of the two handle portions 22 are each arranged in a top view of the connector handle 20 in the circumferential direction between the adapter portions 19a, 19b. In other words, the handle bars 23 are each offset by approximately 90° with respect to the adapter portions 19a, 19b in the circumferential direction of the main body 21. The handle plates 24 are connected vertically to the handle bars 23, so that the handle portions 22 substantially form a T-profile in the top view of the connector handle 20.

Several ribs 26 are formed on a handle surface 25 of the handle plates 24 facing away from the main body 21, which extend over almost the entire width of the handle surface 25 and enable improved haptics and handling of the connector 1.

According to the preferred configuration examples, the connector 1 further comprises a union sleeve/cover element 27. The union sleeve 27 is slid over the connector 1, i.e. the adapter piece 5 and the counter-piece 15, in order to make access to the connector 1 in the connected/screwed state more difficult or to prevent it. In other words, the union sleeve 27 serves as a safeguard against unintentional or inadvertent opening of the connector 1.

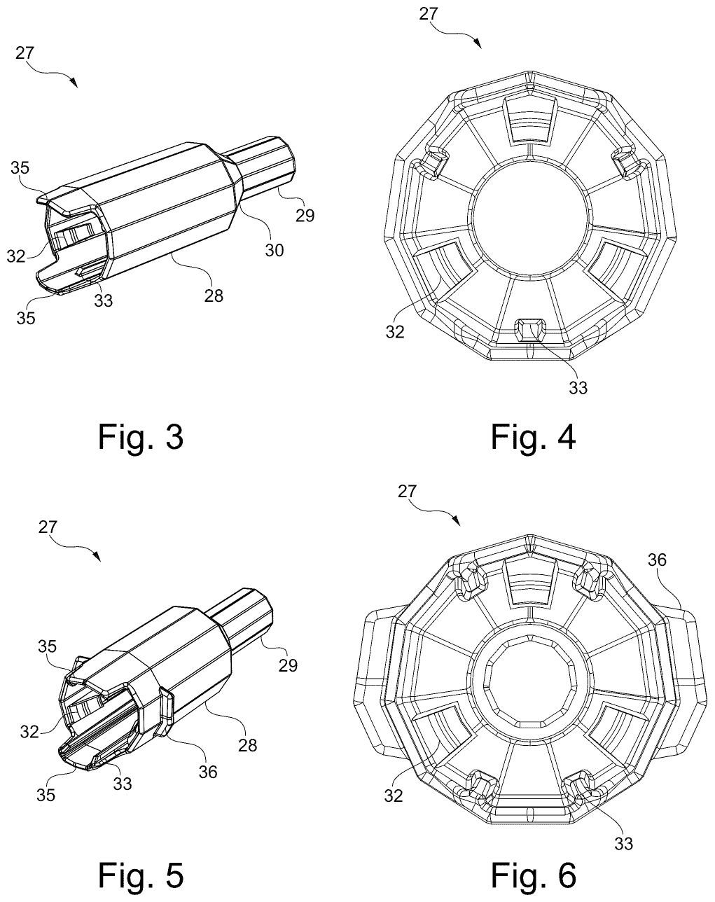

As shown in FIG. 3, the union sleeve 27 has a first sleeve portion 28 with a large diameter and a second sleeve portion 29 with a small diameter. The first sleeve portion 28 and the second sleeve portion 29 are connected to each other via a conical portion 30. In the conical portion 30, three passage openings 31 are formed evenly around the circumference.

Furthermore, the union sleeve 27 has three elastic latching hooks 32 distributed evenly in the circumferential direction on an inner circumferential surface of the first sleeve portion 28 (see FIG. 4). These are formed on a side of the first sleeve portion 28 facing away from the second sleeve portion 29 on the inner circumferential surface and project inward from this surface into the union sleeve 27, i.e. in the direction of the central axis of the union sleeve 27 and in the direction toward the second sleeve portion 29.

If, as shown in FIG. 2, the union sleeve 27 is now pushed over the connector 1 in the connected state, the latching hooks 32 are pressed/pre-tensioned outward in the radial direction by the adapter piece 5 and slide along an outer circumferential surface of the adapter piece 5. When the union sleeve 27 has been slid completely over the connector 1, the latching hooks 32 snap inward in a radial direction and thus engage with a front face of the adapter piece 5 facing the counter-piece 15. It is therefore no longer possible to pull the union sleeve 27 off the connector 1 without destroying it. In other words, the latching hooks 32 of the union sleeve 27 latch with the front face of the adapter piece 5, which permanently prevents access to the connector 1 without having to provide specific latching means on the connector 1.

As shown in FIG. 4, the union sleeve 27 furthermore has three rails/ribs 33 formed evenly over the circumference and in each case in the circumferential direction between the latching hooks 32 on the inner circumferential surface of the first sleeve portion 28. In the connected state of the connector 1 and with the union sleeve 27 fully slid on, the rails 33 are in contact with the adapter piece 5, in particular with a support collar 34 formed on an outer circumferential surface of the adapter piece 5, so that relative movement between the adapter piece 5 and the union sleeve 27 can be reduced in the radial direction.

Furthermore, as shown in FIG. 3, the union sleeve 27 has two diametrically opposite, partially circumferential projections 35 on a side facing away from the second sleeve portion 29. In other words, the two projections 35 are formed on a front face of the union sleeve 27 facing away from the second sleeve portion 29 and project in the axial direction from this front face. As shown in FIG. 1, in the slid-on state of the union sleeve 27, the projections 35 engage in the cavity formed between the handle plates 24. In other words, in the slid-on state of the union sleeve 27, the projections 35 are held between the handle plates 24 in the circumferential direction of the union sleeve 27, so that the union sleeve 27 can no longer be rotated relative to the adapter piece 5 in the slid-on state. In other words, the projections 35, which are in engagement with the connector handle 20, in particular with the handle plates 24 of the handle portions 22, serve as an anti-rotation lock for the union sleeve 27.

FIGS. 5 and 6 show the union sleeve 27 according to a modification of the preferred configuration example. Here, two handle elements 36 in the form of wings projecting from the outer circumferential surface are formed on an outer circumferential surface of the union sleeve 27. In addition, according to the modification of the preferred configuration example, as can be seen in FIG. 8, the rails 33 are no longer formed uniformly in the circumferential direction between the latching hooks 32. Rather, according to the modification of the preferred configuration example, four rails 33 are formed on the inner circumferential surface of the first sleeve portion 28, wherein two rails 33 each are arranged in the region of the partially circumferential projections 35. In other words, according to the modification of the preferred configuration examples, the union sleeve 27 has two pairs of rails 33, so that the rails 33 are diametrically opposite each other in pairs.

In the preferred configuration example described above, the conical part 4 and the sleeve part 10 form the adapter piece 5. That is, the adapter piece 5 has a multi-part/multi-piece configuration. Of course, the adapter piece 5 may also be formed from an integral/one-piece component. In other words, the conical part 4 and the sleeve part 10 may form the adapter piece 5 integrally/in one piece.

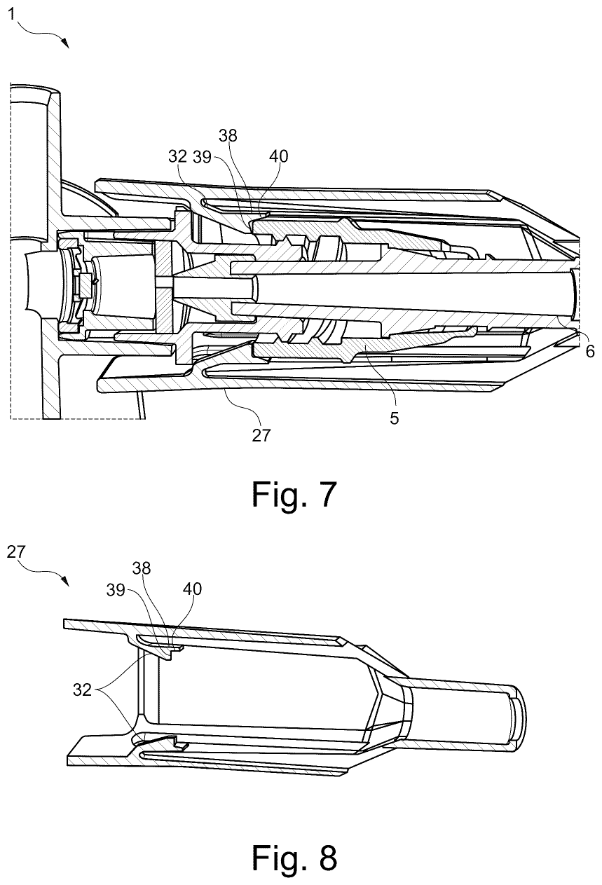

FIGS. 7 and 8 show the connector 1 or respectively the union sleeve 27 according to a further preferred configuration example. Here, the latching hooks 32 are each mounted or hinged at one point on an inner circumferential surface of the union sleeve 27 and project radially inward from this one point into the union sleeve 27.

A latching end portion 38 of the latching hooks 32, i.e. an end portion which latches with the adapter piece 5 in the slid-on state of the union sleeve 27, has an axial latching portion 39 and a radial latching portion 40. The axial latching portion 39 is configured to latch with or rest against the front face of the adapter piece 5 facing the counter-piece 15 in the slid-on state.

The radial latching portion 40, on the other hand, is configured to latch with an outer circumferential surface of the adapter piece 5 in the slid-on state, as can be seen in FIG. 7. In other words, the radial latching portion 40 is configured as an additional latching nose projecting from the respective latching hook 32 in the axial direction, i.e. in the direction toward the tube portion 2, which in the slid-on state of the union sleeve 27 engages behind the outer circumferential surface of the adapter piece 5 and thus fixes the latching hook 32 in the radial direction relative to the adapter piece 5.

In other words, in the union sleeve 27 according to the further preferred configuration example, the latching end portion 38 of the latching hooks 32 is substantially L-shaped with the axial latching portion 39 and the radial latching portion 40. In the slid-on state of the union sleeve 27, the axial latching portion 39 engages with the front face of the adapter piece 5 facing the counter-piece 15, whereas the radial latching portion 40 engages with the outer circumferential surface of the adapter piece 5. In this way, the latching hook 32 can be fixed in a radial direction relative to the adapter piece 5, which makes it possible to prevent the latching hook 32 from sliding radially inward on the front face of the adapter piece 5 facing the counter-piece 15, projecting into the threaded portion 13 and shearing off when a tensile load is applied to the union sleeve 27 in the direction away from the counter-piece 15 (to the right in FIG. 7). The union sleeve 27 or the latching hooks 32 of the union sleeve according to the further preferred configuration example thus has a self-locking effect, so that a higher tensile load can be absorbed without the latching hook 32 shearing off.

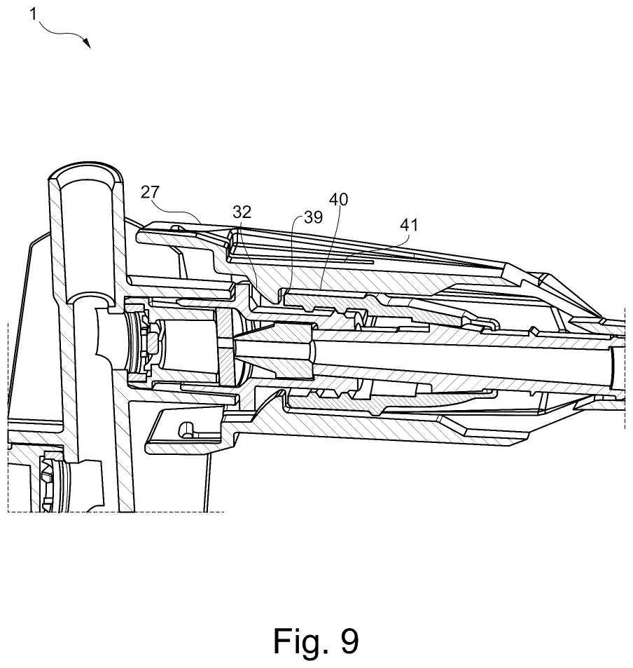

In FIG. 9, the connector 1 or the union sleeve 27 is shown according to a modification of the further preferred configuration example. In this case, the latching hooks 32 are each mounted or hinged at two points on the inner circumferential surface of the union sleeve 27 and project radially inward into the union sleeve 27 between these two points. In order to be able to deflect the latching hooks 32, as mentioned above, when the union sleeve 27 is slid over the adapter piece 5, they need to have a certain elasticity/spring effect. For this purpose, clearances/apertures 41 are formed in a lateral surface of the union sleeve 27 in a circumferential direction laterally next to the latching hooks 32 in the union sleeve 27 according to the modification of the further preferred configuration example. This means that the union sleeve 27 has two apertures 41 extending through the union sleeve 27 for each latching hook 32 in order to ensure the spring effect of the latching hook 32.

As can be seen in FIG. 9, the latching hooks 32 of the union sleeve 27 according to the modification of the further preferred configuration example has the axial latching portion 39, which in the slid-on state engages with the front face of the adapter piece 5 facing the counter-piece 15, and the radial latching portion 40, which in the slid-on state at least partially abuts against the outer circumferential surface of the union sleeve 27. This ensures that the latching hooks 32 of the union sleeve 27 according to the modification of the further preferred configuration example do not slide radially inward into the threaded portion 13 under tensile load. This self-locking action therefore prevents the latching hooks 32 from shearing off.

### LIST OF REFERENCE SIGNS

1 connector
2 tube portion
3 adapter portion
4 conical part
5 adapter piece
6 fluid opening
7 insertion portion
8 first latching portion
8a ramp surface
8b latching surface
9 second latching portion
9a ramp surface
9b latching surface
9c step
10 sleeve part
11 step
12 connection portion
13 threaded portion
14 internal thread
15 counter-piece
16 outer thread
17 inner circumferential surface
18 receiving opening
19 adapter portion
20 connector handle
21 main body
22 handle portion
23 handle bar
24 handle plate
25 handle surface
26 rib
27 union sleeve
28 first sleeve portion
29 second sleeve portion
30 conical portion
31 passage opening
32 latching hook
33 rail/rib
34 support collar
35 partially circumferential projections
36 handle element
37 check valve
38 latching end portion
39 axial latching portion
40 radial latching portion
41 aperture

1.-10. (canceled)

11. A connector for establishing a fluid connection between a first fluid transmission component and a second fluid transmission component, the connector comprising:
an adapter piece comprising an internal thread and a front face;
a counter-piece comprising an outer thread that is screwed to the internal thread of the adapter piece, the front face of the adapter piece facing the counter-piece; and
a union sleeve that is slidable over the adapter piece and the counter-piece, wherein the union sleeve, when in a slid-on state, latches non-detachably with the adapter piece, and wherein the union sleeve comprises a latch that latches in the slid-on state with the front face of the adapter piece.

12. The connector according to claim 11, wherein the connector comprises the first fluid transmission component, the first fluid transmission component comprising a tube portion, and wherein the adapter piece has a sleeve part forming the internal thread and a conical part housed in the sleeve part and connected to the tube portion of the first fluid transmission component.

13. The connector according to claim 11, wherein the connector comprises a connector handle, wherein the union sleeve has projections in an axial direction on an end portion facing the counter-piece, and wherein the projections are in engagement with the connector handle in the slid-on state.

14. The connector according to claim 13, wherein the connector handle has a main body that is circular cylindrical, at least one first adapter portion branching off from the main body and being in fluid connection with the counter-piece, and two handle portions projecting from an outer circumferential surface of the main body, between which the projections of the union sleeve are held in the slid-on state of the union sleeve.

15. The connector according to claim 14, wherein the connector handle comprises a second adapter portion branching off from the main body and being couplable to a further fluid transmission component.

16. The connector according to claim 15, wherein a central axis of the at least one first adapter portion is parallel to a central axis of the second adapter portion.

17. The connector according to claim 11, wherein the latch is pre-tensioned by the connector when the union sleeve is slid onto the connector.

18. The connector according to claim 11, wherein the latch comprises at least one elastic latching hook projecting axially and radially inward from an inner circumferential surface of the union sleeve.

19. The connector according to claim 18, wherein the union sleeve has three latching hooks evenly distributed over a circumference of the union sleeve.

20. The connector according to claim 11, wherein the union sleeve has at least one handle element on an outer circumferential surface of the union sleeve, the at least one handle element configured to be gripped when the union sleeve is slid on by a user.
